(19) United States
(12) Patent Application Publication
(10) Pub. No.: US 2003/0180777 A1
(43) Pub. Date: Sep. 25, 2003

(54) Rapid identification of transcriptional regulatory domains

(76) Inventor: Victor Bartsevich, Albany, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/387,320

(22) Filed: Mar. 11, 2003

Related U.S. Application Data
(60) Provisional application No. 60/365,004, filed on Mar. 12, 2002.

(51) Int. Cl. C12Q 1/68; G01N 33/53; G01N 33/567; C12N 9/64

(52) U.S. Cl. 435/6; 435/7.2; 435/226

(57) ABSTRACT

Compositions and methods for high-throughput assay for transcriptional regulatory domains in mammalian cells are provided. In certain embodiments, libraries of random amino acid sequences are assayed for transcriptional regulatory activity. In additional embodiments, cDNA libraries are assayed. Libraries are fused to a DNA-binding domain that is targeted to a reporter gene, and modulation of expression of the reporter gene is assayed. Accordingly, regulatory domains having both positive and negative transcriptional regulatory activity can be identified.

### CROSS-REFERENCE TO RELATED APPLICATIONS

This application is related to provisional patent application serial No. 60/365,004, filed Mar. 12, 2002, from which priority is claimed under 35 USC §119(e) and which application is incorporated herein by reference in its entirety.

### TECHNICAL FIELD

This disclosure is in the field of molecular biology and medicine. More specifically, it relates to methods of identifying transcriptional regulatory domains, the regulatory domains themselves, and the use of the domains for regulation of gene expression.

### BACKGROUND

Worldwide genome sequencing efforts are providing a wealth of information on the sequence and structure of various genomes, and on the locations of thousands of genes. This genome research is yielding a considerable amount of information on gene products and their functions. In addition, research regarding the location, extent, nature and function of sequences that regulate gene expression, i.e., cis-acting gene regulatory sequences, is also underway. (See, e.g., co-owned WO 01/83732).

Trans-acting factors, also known as transcription factors, have also been studied. Typically, transcription factors have modular structures with distinct DNA-binding and transcriptional regulatory domains. See, e.g., Shastry et al. (1993) Experientia 49:831-835; Vrana et al. (1988) Mol Cell Biol 8(4): 1684-96). These distinct domains can be interchanged, for example to generate fusion polypeptides having specificity for a particular DNA sequence along with a domain that activates or represses transcription. See, e.g., International PCT Publication WO 00/41566. These artificial transcription factors represent powerful tools for basic research and gene therapy.

Naturally-occurring DNA binding domains have been well characterized. Miller et al. (1985) EMBO J. 4:1609-1614; Rhodes et al. (1993) Scientific American February:56-65; and Klug (1999) J. Mol. Biol. 293:215-218; Berg (1992) Proc. Natl. Acad. Sci. USA 89:11,109-11,110. Additionally, it has recently become possible to design DNA-binding polypeptides that can recognize virtually any possible sequence. (See, e.g., WO 00/42219; WO 00/41566; as well as U.S. Pat. Nos. 5,789,538; 6,007,408; 6,013,453; 6,140,081; 6,140,466 and 6,242,568; and PCT publications WO 95/19431, WO 98/54311, WO 96/06166, WO 00/23464; WO 00/27878; WO98/53057; WO98/53058; WO98/53059; and WO98/53060). This has significantly extended the potential applications of designed transcriptional factors.

With regard to transcriptional regulatory domains (i.e., functional domains), several naturally occurring domains have been characterized and used in combination with engineered DNA binding proteins. For instance, known repression domains include a KRAB repression domain from the human KOX-1 protein (see, e.g., Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994); a methyl binding domain protein 2B (MBD-2B) (see, also Hendrich et al. (1999) Mamm Genome 10:906-912 for description of MBD proteins); sequences obtained from a v-ErbA protein (See, Damm, et al. (1989) Nature 339:593-597; Evans (1989) Int. J. Cancer Suppl. 4:26-28; Pain et al. (1990) New Biol. 2:284-294; Sap et al. (1989) Nature 340:242-244; Zenke et al. (1988) Cell 52:107-119; and Zenke et al. (1990) Cell 61:1035-1049); thyroid hormone receptor (TR); SID; MBD1; MBD2; MBD3; MBD4; MBD-like proteins; members of the DNMT family (e.g., DNMT1, DNMT3A, DNMT3B); Rb; MeCP1 and MeCP2) (See, for example, Zhang et al. (2000) Ann Rev Physiol 62:439-466; Bird et al. (1999) Cell 99:451-454; Tyler et al. (1999) Cell 99:443-446; Knoepfler et al. (1999) Cell 99:447-450; and Robertson et al. (2000) Nature Genet. 25:338-342); ROM2 and AtHD2A. (See, for example, Chem et al. (1996) Plant Cell 8:305-321; and Wu et al. (2000) Plant J. 22:19-27).

Activation domains include the HSV VP16 activation domain (see, e.g., Hagmann et al., J. Virol. 71, 5952-5962 (1997)); nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Seifpal et al., EMBO J. 11, 4961-4968 (1992)); p300, CBP, PCAF, SRC1 PvALF, and ERF-2 (See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504); OsGAI, HALF-1, C1, AP1, ARF-5,-6,-7, and -8, CPRF1, CPRF4, MYC-RP/GP, and TRAB1 (See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353); as well as those disclosed in WO 00/41566; WO 01/83793 and PCT/US01/42377.

Despite the fact that a variety of functional domains are known, the activities of many transcriptional regulatory domains are gene specific and/or cell specific. Thus, a particular functional domain may exert its effects only on certain genes and/or only in certain cell types. Accordingly, methods for obtaining gene-specific and/or cell/specific transcriptional regulatory domains would represent a significant advance in the art, as would the gene-specific and cell-specific regulatory domains obtained by such methods.

Furthermore, current methods of screening combinatorial libraries for functional domains are generally carried out in vitro (e.g., phage display) and yield domains that, while active in vitro, are not necessarily able to function similarly in vivo. Thus, methods and compositions that would allow the identification and isolation of transcriptional regulatory domains that are known to be functional in vivo would be of great value.

### SUMMARY

Described herein are methods and compositions that allow for the identification and isolation of transcriptional regulatory domains.

In one aspect, a method of identifying a transcriptional regulatory peptide is disclosed, the method comprising the steps of: (a) introducing a library of expression vectors into cells, wherein the cells comprise a reporter gene and wherein the expression vectors of the library encode proteins comprising: (i) a DNA-binding domain targeted to the reporter gene, and (ii) at least one putative regulatory domain; (b) identifying one or more cells in which expression of the reporter gene is modulated; (c) isolating the expression vector from the cell identified in step (b); and (d) determining the sequence of the putative regulatory domain in the vector isolated in step (c), thereby identifying a transcriptional regulatory peptide. In certain embodiments, the putative regulatory domain comprises a randomized sequence or a cDNA sequence. In any of the methods described herein, the DNA-binding domain can comprise one or more zinc fingers, for example an engineered zinc finger.

Also described herein is a library of polynucleotide sequences, wherein the sequences of the library encode proteins comprising: (a) a DNA-binding domain targeted to a reporter gene, and (b) at least one putative regulatory domain. The libraries may be in the form of expression vectors. In certain embodiments, the putative regulatory domain comprises a randomized sequence or a cDNA sequence. In any of the libraries described herein, the DNA-binding domain can comprise one or more zinc fingers, for example an engineered zinc finger.

In any of the methods or compositions (e.g., libraries) described herein, the reporter gene can be either an endogenous gene or an exogenous gene. Examples of reporter genes include, but are not limited to, those that encode a molecule that is expressed on the surface of the cell, green fluorescent protein (GFP), chloramphenicol acetyl transferase (CAT), luciferase, beta-galactosidase, and/or a protein that confers resistance to antibiotics. In certain embodiments, the reporter gene is stably maintained in the cell, e.g., as an integrated sequence or as an episome. Alternatively, the reporter gene is transiently transfected into the cell. Expression of the reporter gene can be activated or repressed. In one embodiment, the reporter gene is the P2X7 gene.

Further, in any of the methods described herein, identifying one or more cells in which expression of a reporter gene is modulated can comprise analyzing the cells by fluorescence-activated cell sorting (FACS).

Additional embodiments include a method of identifying a transcriptional regulatory peptide, wherein the method comprises the steps of contacting a population of cells comprising a reporter gene with a library of expression vectors, wherein each expression vector encodes a protein comprising (i) a first domain comprising a DNA-binding domain which binds to the reporter gene, and (ii) a second domain; identifying a cell in which expression of the reporter gene is modulated; and characterizing the vector from said cell so as to identify the second domain, wherein said second domain encodes a transcriptional regulatory peptide.

In certain embodiments of these methods, the DNA-binding domain comprises at least one zinc finger, e.g., an engineered zinc finger.

In certain embodiments, the second domain is encoded by a randomized sequence. In additional embodiments, the second domain is encoded by a cDNA sequence.

The reporter gene can be an endogenous gene or an exogenous gene. An exogenous gene can be transiently transfected into a cell or stably maintained in a cell, e.g., as a sequence integrated into the cellular genome or as an extrachromosomal episome.

In exemplary embodiments, a reporter gene can encode a molecule that is expressed on the cell surface (e.g., a cell-surface protein) or a product selected from the group consisting of green fluorescent protein (GFP), chloramphenicol acetyl transferase (CAT), luciferase, beta-galactosidase, and a protein that confers resistance to antibiotics. An additional exemplary reporter gene is the P2X7 gene.

In the practice of the disclosed methods, expression of a reporter gene is modulated. Reporter gene expression can be either activated or repressed. Modulation of reporter gene expression can occur in one or more cells in the population and, in certain embodiments, occurs in a plurality of cells.

Techniques for identifying cells in which reporter gene expression is modulated are known to those of skill in the art and include, for example, analysis of RNA levels, analysis of proteins levels, analysis of enzymatic activity, analysis of cellular phenotype. Exemplary methods include real-time PCR (TaqMan®), ELISA, flow cytometry and fluorescence-activated cell sorting (FACS).

Characterization of a vector from a cell includes methods to determine the structure and properties of the vector, including but not limited to restriction digestion, sedimentation, affinity purification and determination of coding capacity e.g., by coupled transcription/translation. An exemplary method for characterizing a vector is to determine its nucleotide sequence, from which an amino acid sequence of one or more encoded polypeptides can be derived.

Also provided are libraries of polynucleotide sequences, wherein each member of the library comprises a sequence encoding a DNA-binding domain which binds to a reporter gene, and a randomized sequence. Also provided are libraries of polynucleotide sequences, wherein each member of the library comprises a sequence encoding a DNA-binding domain which binds to a reporter gene, and a cDNA sequence.

Also described herein is a population of cells, wherein at least one member of the population comprises a member of any of the libraries described herein.

As will become apparent, preferred features and characteristics of the aspects described herein are applicable to any other aspects.

### BRIEF DESCRIPTION OF THE DRAWINGS

FIG. 1 shows flow cytometric analysis of Neuro-2A cells that had been infected with a retrovirus expressing a ZFP/KOX-1 fusion protein targeted to the P2X7 gene (right panel), and control uninfected cells (left panel). In each panel, the left-most trace (closer to the ordinate) represents cells that were exposed to YO-PRO-1 (Yo-Pro-1) in the absence of BzATP, and the right-most trace represents cells that were exposed to YO-PRO-1 in the presence of BzATP.

FIG. 2 shows levels of P2X7 mRNA (normalized to GAPDH mRNA) in Neuro-2A cells that had been infected with a retrovirus expressing a ZFP/KOX-1 fusion protein targeted to the P2X7 gene (right bar), and control uninfected cells (left bar).

### DETAILED DESCRIPTION

Disclosed herein are methods for high-throughput screening of libraries of random peptides and/or cDNAs, to identify new transcriptional regulatory polypeptides. The methods and compositions allow for the identification of both positive and negative transcriptional regulatory polypeptides. In addition, transcriptional regulatory polypeptides that are specific for a particular promoter sequence (e.g., gene-specific transcriptional regulators) and/or for a particular cell type are identified using the methods and compositions disclosed herein.

Briefly, the methods described herein make use of libraries of randomized peptide sequences linked to a DNA-binding domain, or libraries of cDNAs linked to a DNA-binding domain. The libraries are screened for transcriptional activity (positive or negative), and library members having the desired activity are selected and serve as a source of new, gene-specific and/or cell-specific regulatory domains.

For screening, libraries can be constructed in plasmid or viral vectors and used for transient transfection or stable transduction of a recipient cell expressing any suitable reporter. The reporter can be an endogenous cellular gene or can be, e.g., cotransfected with the library. Alternatively, stable cell lines containing a reporter gene can be generated. In certain embodiments, the DNA-binding portion of the polypeptides encoded by the library is engineered to bind at or near the reporter gene. Following transfection of the library, polypeptides encoded by the library bind to the reporter gene, and levels of expression of the reporter gene in individual cells is evaluated in a high-throughput fashion, for example by fluorescence-activated cell sorting (FACS) analysis or by using positive or negative selection methods. Cells exhibiting altered (e.g., higher or lower) levels of transcription of the reporter gene (as compared to control cells), or cells surviving a selection, are then collected and the transfected nucleic acid in these cells is analyzed (e.g., by nucleotide sequence analysis) to identify the regulatory domain. A cell in which expression of the reporter gene is increased or decreased contains a library member whose random peptide or cDNA portion encodes a transcriptional activation or repression domain, respectively. One or more steps of the method (e.g., selection and/or sorting) can be optionally repeated, if necessary, in order to analyze single clones.

In one embodiment, a negative selection is used to screen for repression domains. More specifically, recipient cells comprise an activity that can be induced to promote cell death. For example, recipient cells express a “death gene” whose product, optionally in the presence of an inducer, leads to death of the cell e.g. by apoptosis. To screen for repression domains, such cells are transfected with a library (as described above) and optionally exposed to inducing substance or subjected to inducing activity. Cells in which expression of the death gene is inhibited will survive the presence of the inducer, and the transfected nucleic acids from surviving cells encode repression domains. In an exemplary embodiment, the death gene encodes a purinergic receptor (e.g., the P2X7 cell surface receptor) and the cells are exposed to ATP or BzATP. See, for example, Coutinho-Silva et al. (1999) Am. J. Physiol. May;276(5 Pt 1):C1139-1147; Virgino et al. (1 999) J. Physiol. September 1;519 Pt 2:335-346; Le Feuvre et al. (2002) J. Biol. Chem. 277(5):3210-3218; Brough et al. (2002) Mol. Cell. Neurosci. 19(2):272-280.

Thus, using the methods and compositions disclosed herein, polypeptides (and polynucleotides encoding these polypeptides) having a desired transcriptional regulatory function (that would not be readily identified, e.g. by sequence homology) can be identified and isolated. The desired transcriptional regulatory activity can be either positive or negative, and can be specific to a particular gene or group of genes, as well as specific to a particular cell type, depending on the cell in which the assay is conducted. The disclosed screening methods are facilitated by the use of engineered DNA binding proteins that bind to predetermined DNA sequences. Thus, by targeting randomized peptides or cDNA libraries to specific DNA sequences, novel functional domain polypeptides are identified.

In preferred embodiments, screening and/or selection is conducted in mammalian cells (e.g., cultured cell lines). The disclosed methods and compositions advance the art by allowing high-throughput screening for new transcriptional activation and/or repression domains (i.e., functional domains) in mammalian cells. The disclosed methods and compositions are superior to previous in vitro methods for assay of combinatorial libraries such as, for example, phage display; because they do not require knowledge about the interaction of a putative functional domain with a binding partner, and because the domains obtained by practice of the disclosed methods are selected based on their ability to function within mammalian cells.

Definitions

Use of the disclosed compositions and practice of the disclosed methods employ, unless otherwise indicated, conventional techniques in molecular biology, biochemistry, chromatin structure and analysis, combinatorial chemistry, cell culture, recombinant DNA and related fields as are within the skill of the art. These techniques are fully explained in the literature. See, for example, Sambrook et al. MOLECULAR CLONING: A LABORATORY MANUAL, Third edition, Cold Spring Harbor Laboratory Press, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Harlow & Lane, eds. ANTIBODIES: A LABORATORY MANUAL, Cold Spring Harbor Laboratories Press, 1988.

The terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T.

An “exogenous molecule” is a molecule that is not normally present in a cell, but can be introduced into a cell by one or more genetic, biochemical or other methods. Normal presence in the cell is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotien, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases,, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., protein or nucleic acid (i.e., an exogenous gene), providing it has a sequence that is different from an endogenous molecule. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous molecule” is one that is normally present in a particular cell at a particular developmental stage under particular environmental conditions. For example, an endogenous nucleic acid can comprise a chromosome, the genome of a mitochondrion, chloroplast or other organelle, or a naturally occurring episomal nucleic acid. Additional endogenous molecules can include proteins, for example, transcription factors and components of chromatin remodeling complexes.

A “fusion molecule” is a molecule in which two or more subunit molecules are linked, preferably covalently. The subunit molecules can be the same chemical type of molecule, or can be different chemical types of molecules. Examples of the first type of fusion molecule include, but are not limited to, fusion polypeptides (for example, a fusion between a zinc finger DNA-binding domain and a peptide sequence) and fusion nucleic acids (for example, a nucleic acid encoding the fusion polypeptide described supra). Examples of the second type of fusion molecule include, but are not limited to, a fusion between a triplex-forming nucleic acid and a polypeptide, and a fusion between a minor groove binder and a nucleic acid.

A “gene,” for the purposes of the present disclosure, includes a DNA region encoding a gene product (see infra), as well as all DNA regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. A reporter gene is a gene whose expression is measured as part of an assay.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a MRNA. Gene products also include RNAs that are modified, by processes such as capping, polyadenylation, methylation, and editing, and proteins modified by, for example, methylation, acetylation, phosphorylation, ubiquitination, ADP-ribosylation, myristilation, and glycosylation.

A “zinc finger binding protein” or “zinc finger binding domain” is a protein or segment within a larger protein that binds DNA, RNA and/or protein in a sequence-specific manner via one or more zinc fingers. A zinc finger is a polypeptide domain of approximately 30 amino acid residues which has sequence-specific binding ability and whose three-dimensional structure is stabilized through coordination of a zinc (or related) ion. Such a protein or domain can be further characterized as DNA-binding, RNA-binding or protein-binding. The term zinc finger binding protein is often abbreviated as zinc finger protein or ZFP.

“Transcriptional regulation” or “transcriptional control” refers to the modulation of gene expression at the level of mRNA formation, including but not limited to transcription complex formation, transcription initiation, transcription elongation, transcriptional pausing, splicing, transcriptional termination, polyadenylation and 3′ end maturation. Transcriptional regulation may be positive (e.g., resulting in gene activation) or negative (e.g., resulting in gene repression).

“Gene activation” and “augmentation of gene expression” refer to any process that results in an increase in production of a gene product. A gene product can be either RNA (including, but not limited to, mRNA, rRNA, tRNA, and structural RNA) or protein. Accordingly, gene activation includes those processes that increase transcription of a gene and/or translation of a mRNA. Examples of gene activation processes which increase transcription include, but are not limited to, those which facilitate formation of a transcription initiation complex, those which increase transcription initiation rate, those which increase transcription elongation rate, those which increase processivity of transcription and those which relieve transcriptional repression (by, for example, blocking the binding of a transcriptional repressor). Gene activation can constitute, for example, inhibition of repression as well as stimulation of expression above an existing level. Examples of gene activation processes that increase translation include those, which increase translational initiation, those that increase translational elongation, and those that increase mRNA stability. In general, gene activation comprises any detectable increase in the production of a gene product, preferably an increase in production of a gene product by about 2-fold, more preferably from about 2- to about 5-fold or any integer therebetween, more preferably between about 5- and about 10-fold or any integer therebetween, more preferably between about 10- and about 20-fold or any integer therebetween, still more preferably between about 20- and about 50-fold or any integer therebetween, more preferably between about 50- and about 100-fold or any integer therebetween, more preferably 100-fold or more.

“Gene repression” and “inhibition of gene expression” refer to any process that results in a decrease in production of a gene product. A gene product can be either RNA (including, but not limited to, mRNA, rRNA, tRNA, and structural RNA) or protein. Accordingly, gene repression includes those processes that decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes that decrease translation include those that decrease translational initiation, those that decrease translational elongation and those that decrease mRNA stability. Transcriptional repression includes both reversible and irreversible inactivation of gene transcription. In general, gene repression comprises any detectable decrease in the production of a gene product, preferably a decrease in production of a gene product by about 2-fold, more preferably from about 2- to about 5-fold or any integer therebetween, more preferably between about 5- and about 10-fold or any integer therebetween, more preferably between about 10- and about 20-fold or any integer therebetween, still more preferably between about 20- and about 50-fold or any integer therebetween, more preferably between about 50- and about 100-fold or any integer therebetween, more preferably 100-fold or more. Most preferably, gene repression results in complete inhibition of gene expression, such that no gene product is detectable.

The term “modulate” refers to a change in the quantity, degree or extent of a function. Thus, “modulation” of gene expression includes both gene activation and gene repression. Modulation of gene expression in, for example, a transfected cell, can be assessed with respect to a control cell, e.g., an untransfected cell.

Modulation can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP (see, e.g., Mistili & Spector, (1997) Nature Biotechnology 15:961-964); changes in signal transduction; changes in phosphorylation and dephosphorylation; changes in receptor-ligand interactions; changes in concentrations of second messengers such as, for example, cGMP, cAMP, IP₃, and Ca²⁺; changes in cell growth, changes in neovascularization, and/or changes in any functional effect of gene expression. Measurements can be made in vitro, in vivo, and/or ex vivo. Such functional effects can be measured by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays; changes in intracellular second messengers such as cGMP and inositol triphosphate (IP₃); changes in intracellular calcium levels; cytokine release, and the like.

A “regulatory domain” or “functional domain” refers to a protein or a polypeptide sequence that has transcriptional modulation activity, or that is capable of interacting with proteins and/or protein domains that have transcriptional modulation activity.

“Eucaryotic cells” include, but are not limited to, fungal cells (such as yeast), insect cells, plant cells, animal cells (e.g., avian, teleost, amphibian, reptilian), mammalian cells (e.g., bovine, equine, ovine, porcine, canine, feline) and human cells.

The terms “operative linkage” and “operatively linked” are used with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. An operatively linked transcriptional regulatory sequence is generally joined in cis with a coding sequence, but need not be directly adjacent to it. For example, an enhancer can constitute a transcriptional regulatory sequence that is operatively-linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a transcriptional activation domain (or functional fragment thereof), the ZFP DNA-binding domain and the transcriptional activation domain (or functional fragment thereof) are in operative linkage if, in the fusion polypeptide, the ZFP DNA-binding domain portion is able to bind its target site and/or its binding site, while the transcriptional activation domain (or functional fragment thereof) is able to activate transcription.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide analogues or substitutions. Methods for determining the function of a nucleic acid (e.g., coding function, ability to hybridize to another nucleic acid) are well known in the art. Similarly, methods for determining protein function are well known. For example, the DNA-binding function of a polypeptide can be determined, for example, by filter-binding, electrophoretic mobility-shift, or immunoprecipitation assays. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

The term “recombinant,” when used with reference to a cell, indicates that the cell comprises an exogenous nucleic acid, or expresses a peptide or protein encoded by an exogenous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette,” “expression cassette” or “expression vector” is a nucleic acid construct, generated recombinantly or synthetically, that has control elements that are capable of effecting expression of a gene that is operatively linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired gene product such as, for example, a fusion protein) and a promoter. Additional factors necessary or helpful in effecting expression can also be present. For example, an expression cassette can also include nucleotide sequences that encode a signal sequence that directs secretion of an expressed protein from the host cell. Transcription termination signals, enhancers, and other nucleic acid sequences that influence gene expression can also be included in an expression cassette.

The term “naturally occurring,” as applied to an object, means that the object has not been modified (e.g., by recombinant methods) from the form in which it exists in nature.

The terms “polypeptide,” “peptide” and “protein” are used interchangeably to refer to a polymer of amino acid residues. The term also applies to amino acid polymers in which one or more amino acids are chemical analogues of a corresponding naturally occurring amino acids, and to amino acid polymers that contain non-peptide or modified peptide linkages.

As used herein, “random peptide” and “random peptide sequence” refer to amino acid polymers comprising two or more amino acid monomers (including amino acid analogues) and constructed by a stochastic or random process. A random peptide can include framework or scaffolding motifs, which may comprise invariant sequences.

As used herein “random peptide library” refers to a set of polynucleotide sequences that encode a set of polypeptides comprising random peptide sequences, and to the set of polypeptides (comprising random peptide sequences) encoded by those polynucleotide sequences. Thus, for example, a random peptide library can comprise a plurality of fusion proteins containing random peptide sequences; and/or a plurality of polynucleotides encoding such proteins.

A “subsequence” or “segment” when used in reference to a nucleic acid or polypeptide refers to a sequence of nucleotides or amino acids that comprise a part of a longer sequence of nucleotides or amino acids (e.g., a polypeptide), respectively.

The term “antibody” as used herein includes antibodies obtained from both polyclonal and monoclonal preparations, as well as, the following: (i) hybrid (chimeric) antibody molecules (see, for example, Winter et al. (1991) Nature 349:293-299; and U.S. Pat. No. 4,816,567); (ii) F(ab′)2 and F(ab) fragments; (iii) Fv molecules (noncovalent heterodimers, see, for example, Inbar et al. (1972) Proc. Natl. Acad. Scl. USA 69:2659-2662; and Ehrlich et al. (1980) Biochem 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239:1534-1536; and U.K. Patent Publication No. GB 2,276,169, published 21 September 1994); (vii) Mini-antibodies or minibodies (i.e., sFv polypeptide chains that include oligomerization domains at their C-termini, separated from the sFv by a hinge region; see, e.g., Pack et al. (1992) Biochem 31:1579-1584; Cumber et al. (1992) J. Immunology 149B:120-126); and, (vii) any functional fragments obtained from such molecules, wherein such fragments retain specific-binding properties of the parent antibody molecule.

“Specific binding” between an antibody or other binding agent and an antigen, or between two binding partners (e.g., a DNA-binding polypeptide and its binding sequence), means that the dissociation constant for the interaction is less than 10⁻⁶ M. Preferred antibody/antigen or binding partner complexes have a dissociation constant of less than about 10⁻⁷ M, and preferably 10⁻⁸ M to 10⁻⁹ M or 10⁻¹⁰ M or lower.

Fusion Molecules

The high-throughput screening methods described herein typically involve creating a fusion between a DNA-binding domain and a putative regulatory domain (e.g., a random peptide sequence or a cDNA) to be tested for activity. Polynucleotides encoding such fusions are also useful. In additional embodiments, the methods disclosed herein involve fusions between a DNA-binding domain and one or more functional domains (or polynucleotides encoding such fusions). Fusion molecules can be readily designed and constructed so that the putative regulatory polypeptide is brought into proximity with a sequence in a target gene (e.g., a reporter gene) that is bound by the DNA-binding domain. This can be achieved, for example, by using a DNA-binding domain of known binding specificity for the target gene or by engineering a DNA-binding domain to bind the target gene. Any transcriptional regulatory function of the putative regulatory domain (e.g., activation or repression) is then exerted on the target gene.

A. DNA-Binding Domains

A DNA-binding domain portion of the fusion molecule can comprise any molecular entity capable of sequence-specific binding to DNA. Binding to DNA can be mediated by electrostatic interactions, hydrophobic interactions, or any other type of chemical interaction. Examples of moieties that can comprise part of a DNA-binding domain include, but are not limited to, minor groove binders, major groove binders, antibiotics, intercalating agents, peptides, polypeptides, oligonucleotides, and nucleic acids. An example of a DNA-binding nucleic acid is a triplex-forming oligonucleotide.

Minor groove binders include substances that, by virtue of their steric and/or electrostatic properties, interact preferentially with the minor groove of double-stranded nucleic acids. Certain minor groove binders exhibit a preference for particular sequence compositions. For instance, netropsin, distamycin and CC-1065 are examples of minor groove binders that bind specifically to AT-rich sequences, particularly runs of A or T. See, also, WO 96/32496.

Many antibiotics are known to exert their effects by binding to DNA. Binding of antibiotics to DNA is often sequence-specific or exhibits sequence preferences. Actinomycin, for instance, is a relatively GC-specific DNA binding agent.

In a preferred embodiment, a DNA-binding domain is a polypeptide (or a polynucleotide encoding a polypeptide). Certain peptide and polypeptide sequences bind to double-stranded DNA in a sequence-specific manner. For example, transcription factors participate in transcription initiation by RNA Polymerase II through sequence-specific interactions with DNA in the promoter and/or enhancer regions of genes. Defined regions within the polypeptide sequence of various transcription factors have been shown to be responsible for sequence-specific binding to DNA. See, for example, Pabo et al. (1992) Ann. Rev. Biochem. 61:1053-1095 and references cited therein. These regions include, but are not limited to, motifs known as leucine zippers, helix-loop-helix (HLH) domains, helix-turn-helix domains, zinc fingers, β-sheet motifs, steroid receptor motifs, bZIP domains, homeodomains, AT-hooks and others. The amino acid sequences of these motifs are known and, in some cases, amino acids that are critical for sequence specificity have been identified. Polypeptides involved in other process involving DNA, such as replication, recombination and repair, will also have regions involved in specific interactions with DNA. Peptide sequences involved in specific DNA recognition, such as those found in transcription factors, can be obtained through recombinant DNA cloning and expression techniques or by chemical synthesis, and can be attached to other components of a fusion molecule by methods known in the art.

In a more preferred embodiment, a DNA-binding domain comprises a zinc finger DNA-binding domain. See, for example, Miller et al. (1985) EMBO J. 4:1609-1614; Rhodes et al. (1993) Scientific American February:56-65; and Klug (1999) J. Mol. Biol. 293:215-218. In one embodiment, a target site for a zinc finger DNA-binding domain (i.e., a nucleotide sequence to which a zinc finger binding domain exhibits specific binding) is identified according to site selection rules disclosed in co-owned WO 00/42219 and U.S. Pat. No. 6,453,242. ZFP DNA-binding domains are engineered (e.g., designed and/or selected) to recognize a particular target site as described in co-owned WO 00/42219 and WO 00/41566; as well as, for example, U.S. Pat. Nos. 5,789,538; 6,007,408; 6,013,453; 6,140,081; 6,140,466 and 6,242,568; and PCT publications WO 95/19431, WO 98/54311, WO 00/23464; WO 00/27878; WO98/53057; WO98/53058; WO98/53059; and WO98/53060.

Certain DNA-binding domains are capable of binding to DNA that is packaged in nucleosomes. See, for example, Cordingley et al. (1987) Cell 48:261-270; Pina et al. (1990) Cell 60:719-731; and Cirillo et al. (1998) EMBO J. 17:244-254. Certain zinc finger-containing proteins such as, for example, members of the nuclear hormone receptor superfamily, are capable of binding DNA sequences packaged into chromatin. These include, but are not limited to, the glucocorticoid receptor and the thyroid hormone receptor. Archer et al. (1992) Science 255:1573-1576; Wong et al. (1997) EMBO J. 16:7130-7145. Other DNA-binding domains, including certain zinc finger-containing binding domains, can require more accessible DNA for binding. In these cases, accessible regions in cellular chromatin can be identified as described, for example, in co-owned International PCT Publications WO 01/83732 and WO 01/83751. A DNA-binding domain is then designed and/or selected to bind to a target site within the accessible region.

B. Randomized Peptides

In certain embodiments, a fusion molecule includes a portion containing randomized peptide sequence, to be screened for its transcriptional regulatory capability. The randomized peptide sequence can be of any length, but is typically between about 5 and 100 amino acids in length (or an integer therebetween), preferably between about 10 and 50 amino acids in length (or an integer therebetween), preferably between about 10 and 30 amino acids in length (or an integer therebetween), or between about 10 and 20 amino acids in length (or any integer value therebetween). The randomized polypeptide may exhibit homology to known functional domains or, alternatively, may not exhibit such homology.

Methods for the combinatorial synthesis of random peptide sequences are known in the art. In addition, polynucleotide sequences can be synthesized to encode random peptide sequences by randomization of the nucleotide sequence of all or a portion of the polynucleotide. Such methods are well-known to those of skill in the art.

C. cDNAs

In certain embodiments, libraries are constructed in which individual members of a cDNA library are fused to a DNA-binding domain that binds a sequence in a target gene. The DNA-binding domain can be naturally-occurring, or engineered.

Methods for the construction of cDNA libraries, and for construction of libraries comprising a plurality of cDNAs fused to a common sequence (such as, for example, one encoding a DNA-binding domain) are known in the art. See, for example, Sambrook et al., supra and Ausubel et al., supra.

Moreover, individual members of the aforementioned libraries can comprise intact cDNA sequences or cDNA sequences that have been modified (e.g., by insertion, deletion or nucleotide substitution) to generate additional sequence diversity. Furthermore, individual members of the library can comprise sequences, either total or partial, from more than one cDNA molecule. Additional techniques useful for generating diversity in cDNA sequences are described, for example, in U.S. Pat. Nos. 5,498,531; 6,132,970; 6,165,793; 6,287,862; 6,319,714 and 6,344,356.

D. Construction of Fusion Molecules

Fusion molecules are constructed by methods of cloning and biochemical conjugation that are well known to those of skill in the art. In certain embodiments, fusion molecules comprise a polypeptide DNA-binding domain and a randomized polypeptide. In other embodiments, the fusion molecule comprises a polynucleotide encoding a DNA-binding domain and a polynucleotide encoding a randomized polypeptide. Fusion molecules may also be comprised of polypeptide-polynucleotide hybrids. Fusion molecules also optionally comprise nuclear localization signals (such as, for example, that from the SV40 T-antigen) and epitope tags (such as, for example, FLAG, myc and hemagglutinin). Fusion proteins (and nucleic acids encoding them) are preferably designed such that the translational reading frame is preserved among the components of the fusion.

Fusions between a polypeptide component on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al. (2000) Proc. Natl. Acad. Sci. USA 97:3930-3935.

The fusion molecules disclosed herein comprise a DNA-binding domain that binds to a target site. In certain embodiments, the target site is present in an accessible region of cellular chromatin. Accessible regions can be determined as described in co-owned International Publication WO 01/83732. If the target site is not present in an accessible region of cellular chromatin, one or more accessible regions can be generated as described in co-owned International Publication WO 01/83793. In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3). Cordingley et al. (1987) Cell 48:261-270; Pina et al. (1990) Cell 60:719-731; and Cirillo et al. (1998) EMBO J. 17:244-254.

Identification of gene-specific regulatory domains can be achieved using the methods described herein, by targeting cDNA-encoded polypeptide sequences or random polypeptide sequences to a specific target gene, by virtue of a fused DNA binding domain. The nature of the regulatory domain can be determined by assaying for modulation of expression of the target gene. Modulation of target gene expression can be in the form of increased expression (thereby identifying an activation domain) or decreased expression (thereby identifying a repression domain).

Identification of cell-specific regulatory domains can be achieved by assaying a library in two or more different cell types, and identifying library members whose activity is restricted to a chosen cell type.

Preparation of Libraries

Described herein are methods of screening transcriptional regulatory domains from cDNA or combinatorial peptide libraries. As used herein, the term “library” refers to a pool of DNA fragments encoding peptides that have been introduced into a cloning vector, or to the collection of peptide sequences themselves. The peptide sequences can be random sequences, non-random sequences (e.g., encoded by cDNA) or a combination of the two.

It is now possible, through synthetic chemistry or molecular biology, to generate libraries of complex polymers, with many subunit permutations. Such libraries can take a number of forms, including, but not limited to: random peptides, which can be synthesized on plastic pins (Geysen et al., 1987, J. Immunol. Meth. 102:259-274), beads (Lam et al., 1991, Nature 354:82-84) or in a soluble form (Houghten et al., 1991, Nature 354:84-86) or expressed on the surface of viral particles (Cwirla et al., 1990, Proc. Natl. Acad. Sci. USA 87:6378-6382; Kay et al., 1993, Gene 128:59-65; Scott and Smith, 1990, Science 249:386-390); nucleic acids (Ellington and Szostak, 1990, Nature 346:818-822; Gao et al., 1994, Proc. Natl. Acad. Sci. USA 91:11207-11211; Tuerk and Gold, 1990, Science 249:505-510); and small organic molecules (Gordon et al., 1994, J. Med. Chem. 37:1385-1401).

Such libraries, and other types of library known to those of skill in the art, can be used to provide a portion of a fusion molecule (i.e. a putative regulatory domain) that can be screened for its transcriptional regulatory potential. Using fusion molecules comprising a DNA-binding domain having known specificity and a putative regulatory domain, the ability of the putative regulatory domain to activate or repress transcription can be readily assessed, as described further herein.

A. DNA Libraries

The libraries of nucleic acids that are screened using the methods described herein can be natural cDNA or genomic libraries or can be combinatorial libraries in which one or more positions is varied in a systematic manner between library members. Some libraries have members that encode short random peptides about 5-100 amino acids long, preferably 10-30 amino acids long. Other libraries can encode variant forms of a naturally occurring protein. Libraries are constructed by cloning a polynucleotide that contains the variable region of library members (and any spacers and nonvariable framework determinants) into a selected cloning site. Using known recombinant DNA techniques (see generally, Sambrook et al., Molecular Cloning, A Laboratory Manual, 3rd ed., Cold Spring Harbor Laboratory Press, Cold Spring Harbor, N.Y., 2001, incorporated by reference in its entirety for all purposes), a polynucleotide or oligonucleotide can be constructed which, inter alia, removes unwanted restriction sites and adds desired ones, reconstructs the correct portions of any sequences which have been removed (such as a correct signal sequence, for example), inserts the spacer and/or conserved framework residues, if any, and corrects the translation frame (if necessary) to produce a fusion protein. A portion of the polynucleotide generally contains one or more variable region domain(s) and another portion contains/encodes spacer and/or framework residues. The sequences are ultimately expressed as peptides (with or without spacer or framework residues). The variable region domain of the polynucleotide comprises the source of the library.

The size of the library varies according to the number of variable codons, and hence the size of the peptides, which are desired. Generally the library will be at least about 10⁴ or 10⁶ members, usually at least 10⁷, and typically 10⁸ or more members. For example, given current transformation efficiencies (in the range of 20-100%), the ability to recover about one prokaryotic colony per sorted eucaryotic cell, and a practical limit of FACS sorting (10⁷-10⁸ cells), it is possible to screen libraries having a complexity of 10⁶-10⁷ members with reasonable confidence, a level sufficient to clone low-abundance cDNAs.

Any of the libraries described herein can be cloned into plasmid or viral vectors and used to stably or transiently transfect a host cell or cell line.

B. Peptide Libraries

Combinatorial peptide libraries can also be created, for example via expression in bacteriophage. Synthetic oligonucleotides, fixed in length, but with multiple unspecified codons can be cloned into genes III, VI, or VIII of bacteriophage M13 where they are expressed as a plurality of peptide:capsid fusion proteins. Methods used in the construction of these libraries, often referred to as random peptide libraries, can be used for construction of the randomized portion of the fusion molecules described herein. Random peptide libraries have successfully yielded peptides that bind to the Fab site of antibodies (Cwirla et al., 1990, Proc. Natl. Acad. Sci. USA 87:6378-6382; Scott and Smith, 1990, Science 249:386-390), cell surface receptors (Doorbar and Winter, 1994, J. Mol. Biol. 244:361-369; Goodson et al., 1994, Proc. Natl. Acad. Sci. USA 91:7129-7133), cytosolic receptors (Blond-Elguindi et al., 1993, Cell 75:717-728), intracellular proteins (Daniels and Lane, 1994, J. Mol. Biol. 243:639-652; Dedman et al., 1993, J. Biol. Chem. 268:23025-23030; Sparks et al., 1994, J. Biol. Chem. 269:23853-23856), DNA (Krook et al., 1994, Biochem. Biophys. Res. Comm. 204:849-854), and many other targets (Winter, 1994, Drug Dev. Res. 33:71-89).

Additional methods for generating randomized sequences are disclosed, for example, in U.S. Pat. Nos. 5,288, 514; 5,359,115 5,362,899; PCT publications WO 91/07087; WO 92/10092; WO 93/09668; WO 93/20242; WO 94/08051; WO 01/18036; Kerr et al. (1993) J. Amer. Chem. Soc. 115:252; Chen et al. (1994) J. Amer. Chem. Soc. 116: 2661 and Blondelle et al. (1995) Trends Anal. Chem. 14:83; the disclosures of which are incorporated by reference in their entireties.

Screening of Libraries

Once prepared or otherwise obtained, the libraries described herein are screened for modulation of transcription of the reporter gene to which the DNA-binding domain of the fusion molecule is known to bind. The libraries contain sequences encoding a DNA-binding domain (e.g., an engineered zinc finger protein with known DNA-binding specificity) linked to a randomized peptide (e.g., 10-30 amino acids) or cDNA.

A. Host Cells and Cell Lines

The libraries described herein are advantageously screened (e.g., evaluated for transcriptional regulatory properties of the library members) following introduction into a host cell or cell line. The host cell can be a higher eukaryotic cell, such as a mammalian cell or a human cell, or a lower eukaryotic cell, such as a yeast cell, or the host cell can be a prokaryotic cell, such as a bacterial cell. Additionally, plant cells can also be used. Non-limiting representative examples of appropriate hosts include bacterial cells, such as E. coli, Bacillus, Streptomyces, Salmonella typhimurium; fungal cells, such as yeast; insect cells such as Drosophila S2 and Spodoptera sf9; animal cells such as HEK293, CHO, COS; U87MG, U2OS or Bowes melanoma; plant cells such as Arabidopsis, Brassica, soybean, etc. The selection of an appropriate host is within the scope of those skilled in the art from the teachings herein.

Vectors are introduced into host cells by any method of transduction, transformation or transfection known in the art; including but not limited to introduction of naked DNA, particle bombardment, biolistics, co-precipitation, electroporation, calcium phosphate-mediated transfer, DEAE-dextran, cell fusion, lipid-mediated transfer, viral infection and direct injection. These engineered host cells can be cultured in conventional nutrient media modified as appropriate for activating promoters, selecting transformants or amplifying genes. The culture conditions, such as temperature, pH and the like, are those previously used with the host cell selected for expression, and will be apparent to the ordinarily skilled artisan.

In certain embodiments, the libraries are screened using cell lines. Cell lines are commercially available or can be readily constructed using methods known in the art. (See, e.g., Sambrook, above). In certain embodiments, the cell line is stably or transiently transfected with a polynucleotide encoding a reporter polypeptide, for example a cell surface protein or GFP. See, e.g., U.S. Pat. No. 6,316,181.

B. Reporter Molecules

It will be apparent that the nature of the library screen depends on the nature of the reporter. The reporter can be any molecule that confers an assayable (e.g., selectable or screenable) property when it is expressed. Non-limiting examples of reporters include the product of an endogenous chromosomal gene or the product of an exogenous reporter gene construct, wherein the gene or construct contains one or more target site(s) for the DNA-binding domain (e.g., engineered ZFP) within or in operative linkage with a sequence encoding an assayable gene product. Exogenous genes include those which are transiently transfected into a cell and those which are stably maintained in a cell, for example as a stable extrachromosomal element or as sequences integrated into the cellular genome.

Non-limiting examples of reporter sequences include those that encode a measurable protein product, as measured in any number of known assays that include both endogenous and exogenous molecules. For example, exogenous reporters such as chloramphenicol acetyl transferase (CAT), fluorescent proteins (such as green fluorescent protein, Chalfie et al. (1994) Science 663:802-805 and its modified derivatives and related fluorescent proteins such as red fluorescent protein); human placental alkaline phosphatase; light generating proteins (e.g., luciferase), and beta-galactosidase can be conveniently measured in assays that include, but are not limited to colorimetric, fluorimetric and enzymatic assays. Reporters can also be selectable markers, for example, markers that confer antibiotic resistance. Exemplary antibiotics that may be used for selection include, but are not limited to actinomycin, ampicillin, chloramphenicol (Wood et al. (1986) CSHSQB 51:1027), methotrexate (Corey et al. (1990) Blood 75:337), erythromycin, gentamycin sulfate, hygromycin, kanamycin, neomycin (Dick et al. (1985) Cell 42:71; Magli et al. (1987) Proc. Natl. Acad. Sci. USA 84:789); penicillin, polymixin B sulfate, streptomycin sulfate, mycophenolic acid (Stuhlmann et al. (1984) Proc. Natl. Acad. Sci. USA 81:7151) and various chemotherapeutic agents (Guild et al. (1988) Proc. Natl. Acad. Sci USA 85:1595; Kane et al. (1989) Gene 84:439, 1989). Exogenous reporters can be co-transfected transiently with the library, or stable cell lines comprising exogenous reporters can be generated.

Further, virtually any endogenous gene can be also be used as a reporter for the methods described herein. In particular, engineered zinc fingers allow targeted binding to any endogenous gene. See, for example, U.S. Pat. Nos. 6,007,988; 6,013,453; 6,140,466; 6,140,081; 6,242,568; 6,453,242 and the following International patent applications: WO 96/06166; WO 98/53057; WO 98/53058; WO 98/53059; WO 98/53060; WO 00/41566 and WO 00/42219. The level at which the targeted endogenous gene is transcribed can be readily monitored by any suitable techniques. Examples of high-throughput methods for assaying gene expression include fluorescence-activated and/or magnetic cell sorting techniques. Such assays are well known and are described in the literature.

Biologically functional assays (both in vitro and in vivo) can also be used to determine the level of transcription of any endogenous gene, for example, by observing phenotypic changes, such as growth, morphology and other functional effects.

In certain aspects, the reporter used for screening is a molecule expressed on the cell surface (e.g., a cell surface protein). A reporter gene encoding a cell surface protein can be introduced into the cell (or cell line) or a reporter gene encoding a surface protein can be endogenous to the cell. As discussed below, the expression level of cell surface proteins (and hence the transcriptional regulatory ability of various members of a library) can be readily screened using well-known techniques, such as fluorescence activated cell sorting (FACS) techniques that make use of antibodies that bind to cells expressing one or more particular cell surface proteins. Non-limiting examples of cell surface proteins that can serve as reporters include receptors; developmental markers (e.g., CD19, CD34, CD40, CD4, CD8, etc.); antigens (e.g., tumor antigens), and the like.

C. One-Hybrid Screening Systems

One-hybrid systems, including for example mammalian systems, yeast systems or variations thereof, can be used in the methods described herein to identify genes encoding proteins that bind to a target, a cis-acting regulatory element or any other short DNA-binding sequence. (See, e.g., Sotiropoulos et al. (1999) Cell 98:159-169). In these one-hybrid systems, detection of the DNA-protein interactions typically occurs while proteins are in their native configuration, and the gene encoding the DNA-binding protein of interest can be available immediately after library screening (M. M. Wang & R. R. Reed, Nature 364: 121-126; C. Alexander et al., Methods 5: 147-155). A yeast one-hybrid assay, commercially available from ClonTech, see Luo et al, “Cloning & analysis of DNA-binding proteins by yeast one-hybrid and one-two-hybird systems” (1996) Biotechniques 20: 564-8, is based on the interaction between a target-specific DNA-binding protein of interest, and a target-independent GAL1 activation domain. Thus, cDNA candidates or random peptide sequences that may encode a regulatory peptide sequence of interest are expressed as fusions with the activation domain.

In a preferred embodiment, a mammalian one-hybrid system is used to assay transcriptional regulation function of a randomized peptide. In this regard, the assay measures activation of a reporter gene in response to binding of the DNA-binding domain of a fusion molecule as described herein to a site positioned upstream of a basal promoter of the reporter gene. The DNA binding domain (e.g., engineered ZFP) is fused to the putative transcriptional regulatory domain. The reporter gene encodes a protein that can be detected, for example by histochemical staining (e.g., beta-galactosidase) or by fluorescence (i.e., Green Fluorescent Protein (GFP)). The ability of the putative transcriptional regulatory domain to modulate transcription of the reporter gene can be tested using a one-hybrid assay system, that is by evaluating the cells for expression of the reporter gene.

Differences in DNA binding specificity exist between different engineered DNA binding molecules and, accordingly, it is feasible to create reporters that are specific for individual DNA binding domains. Further, the principle of such one-hybird systems can be adapted for use in other types of cells, including but not limited to bacteria, and mammalian cultured cells. A bacterial system is inexpensive and easily manipulated, while mammalian cells more closely approximate the in vivo environment in which transcriptional regulatory domains normally operate.

D. FACS Analysis

Cells can be assayed for change in reporter levels (i.e., modulation of expression of a reporter gene) by fluorescence-activated cell sorting (FACS) analysis. In this method, cells are contained in fluid drops or gel drops and passed by a detection apparatus in which the drops are illuminated with an excitation wavelength and a detector measures either fluorescent emission wavelength radiation and/or measures optical density (absorption) at one or more excitatory wavelength(s). The cells suspended in drops are passed across a sample detector under conditions wherein only about one individual cell is present in a sample detection zone at a time. A source illuminates each droplet and a detector, typically a photomultiplier or photodiode, detects emitted radiation. The detector controls gating of the cell in the detection zone into one of a plurality of sample collection regions on the basis of the signal(s) detected. A general description of FACS apparatus and methods in provided in U.S. Pat. Nos. 4,172,227; 4,347,935; 4,661,913; 4,667,830; 5,093,234; 5,094,940; and 5,144,224, incorporated herein by reference. A suitable alternative to conventional FACS is available from One Cell Systems, Inc. Cambridge, Mass.

In certain embodiments, cells comprising one of the libraries described herein can be contacted with an antibody that recognizes a reporter gene product that is expressed on the surface of the cell. This primary antibody can be fluorescently tagged; alternatively, cells that have been exposed to an unlabeled primary antibody can be further exposed to a fluorescent secondary antibody that recognizes the primary antibody. In either case, the cells are then subjected to FACS analysis, using excitation and emission wavelengths appropriate to the fluorescent antibody. Cells with either higher or lower levels of reporter expression (compared to, for example, control untransfected cells) can be collected as sources of, respectively, activation or repression domains.

E. Magnetic Cell Sorting

When a product of a reporter gene is expressed on the surface of a cell, selection can be performed using magnetic cell sorting. In brief, magnetic particles are attached to antibodies that selectively bind to the reporter gene product. These particles are then mixed with cells, such that cells expressing the reporter gene product are bound to the particles via the antibody. The particles, bound to cells expressing the reporter gene product, are collected, for example, by placing a vessel containing the cell-particle mixture in a magnetic field. In one embodiment the cell-partical mixture is introduced into a column that is placed in a strong magnetic field. Cells expressing the reporter gene product, being tagged with the magnetic particles, are retained in the column. Cells in which the reporter gene product is not expressed are washed through the column.

Protocols for magnetic cell sorting are well established and materials are commercially available (for example from Miltenyi Biotec).

F. Use of Purinergic Receptors in High-Throughput Screens for Functional Domains

The P2X7 purinoceptor is a ligand-gated ion channel expressed on the surface of cells of immune and hematopoietic origin. When activated by low concentrations of ATP or the more potent benzoylbenzoyl ATP (BzATP), P2X7 acts as a non-specific channel for small cations. Higher concentrations of agonists stimulate P2X7 to open larger pores permeable to fluorescent DNA-binding dyes such as Ethidium Bromide (EtBr) and Yo-Pro-1 (a less cell-toxic dye).

Prolonged activation of P2X7 results in cell death, by several mechanisms, due specifically to activity of this receptor. Cell death from apoptosis can occur within a few hours of receptor activation. These properties of the P2X7 receptor are described in Coutinho-Silva et al. 1999, supra; Virgino et al. 1999, supra; Le Feuvre et al. 2002, supra; and Brough et al. 2002, supra.

Taking advantage of these properties, the P2X7 gene can be used as a reporter for high-throughput functional screening for activation and repression domains. Thus, in certain embodiments, cells comprising a purinergic receptor such as, for example, the P2X7 gene product, activated by, for example, ATP or BzATP, are transduced or transfected with a library whose members encode a DNA-binding domain targeted to the P2X7 gene fused to a plurality of cDNA or randomized sequences. The cells can then be assayed in several ways. In one embodiment, transfected cells are assayed for dye uptake. Those cells taking up less dye than, e.g., non-transfected cells comprise a library member that contains a putative repression domain; while cells taking up more dye than, e.g., non-transfected cells comprise a library member that contains a putative activation domain. Dye uptake can be measured by any method known in the art including, but not limited to, FACS, light microscopy, fluorescence microscopy and the like.

In another embodiment, cells comprising an activated purinergic receptor (e.g., P2X7) on their surface are transfected with libraries as disclosed herein, and incubated in the presence of one or more P2X7 agonists. Surviving cells are selected; these comprise library members that contain a putative repression domain.

Multiple rounds of screening (e.g., one-hybrid analysis, antibiotic resistance, magnetic cell sorting and/or FACS analysis) can be performed. For example, in the case of transiently transfected cells total DNA can be recovered and used to transform E. coli cells to recover plasmid library DNA and several additional rounds of screening will typically be performed (using pools of plasmid DNA) before single clones will be analyzed. Similarly, in the case of transduction, cell pools will typically be subjected for several rounds of sorting, before single clones will be isolated. Regulatory domains (peptides or cDNA fragments) can be cloned, for example, by PCR from total DNA or purified from vector DNA obtained from single clones.

Polynucleotide and Polypeptide Delivery

In the practice of the methods described herein, fusion molecules comprising a DNA-binding domain and a putative regulatory domain are typically provided to a target cell, in vitro or in vivo, to screen a peptide sequence for transcriptional regulatory function. The fusion molecules can be provided as polypeptides, polynucleotides or combination thereof.

A. Delivery of Polynucleotides Encoding Fusion Proteins

In certain embodiments, the compositions are provided as one or more polynucleotides. In both fusion and non-fusion cases, the nucleic acid can be cloned into intermediate vectors for transformation into prokaryotic or eukaryotic cells for replication and/or expression. Intermediate vectors for storage or manipulation of the nucleic acid or production of protein can be prokaryotic vectors (e.g., plasmids), shuttle vectors, insect vectors, or viral vectors for example. A nucleic acid encoding a fusion protein can also be cloned into an expression vector, for administration to a bacterial cell, fungal cell, protozoal cell, plant cell, or animal cell, preferably a mammalian cell, more preferably a human cell.

To obtain expression of a cloned nucleic acid, it is typically subdloned into an expression vector that contains a promoter to direct transcription. Suitable bacterial and eukaryotic promoters are well known in the art and described, e.g., in Sambrook et al., supra; Ausubel et al., supra; and Kriegler, Gene Transfer and Expression: A Laboratory Manual (1990). Bacterial expression systems are available in, e.g., E. coli, Bacillus sp., and Salmonella. Palva et al. (1983) Gene 22:229-235. Kits for such expression systems are commercially available. Eukaryotic expression systems for mammalian cells, yeast, and insect cells are well known in the art and are also commercially available, for example, from Invitrogen, Carlsbad, Calif. and Clontech, Palo Alto, Calif.

The promoter used to direct expression of the nucleic acid of choice depends on the particular application. For example, a strong constitutive promoter is typically used for expression and purification. In contrast, when a protein is to be used in vivo, either a constitutive or an inducible promoter is used, depending on the particular use of the protein. In addition, a weak promoter can be used, such as HSV TK or a promoter having similar activity. The promoter typically can also include elements that are responsive to transactivation, e.g., hypoxia response elements, Ga14 response elements, lac repressor response element, and small molecule control systems such as tet-regulated systems and the RU-486 system. See, e.g., Gossen et al. (1992) Proc. Natl. Acad. Sci USA 89:5547-5551; Oligino et al.(I998) Gene Ther. 5:491-496; Wang et al. (1997) Gene Ther. 4:432-441; Neering et al. (1996) Blood 88:1147-1155; and Rendahl et al. (1998) Nat. Biotechnol. 16:757-761.

In addition to a promoter, an expression vector typically contains a transcription unit or expression cassette that contains additional elements required for the expression of the nucleic acid in host cells, either prokaryotic or eukaryotic. A typical expression cassette thus contains a promoter operably linked, e.g., to the nucleic acid sequence, and signals required, e.g., for efficient polyadenylation of the transcript, transcriptional termination, ribosome binding, and/or translation termination. Additional elements of the cassette may include, e.g., enhancers.

The particular expression vector used to transport the genetic information into the cell is selected with regard to the intended use of the resulting polypeptide, e.g., expression in mammalian cells, plants, animals, bacteria, fungi, protozoa etc. Standard bacterial expression vectors include plasmids such as pBR322, pBR322-based plasmids, pSKF, pET23D, and commercially available fusion expression systems such as GST and LacZ. Epitope tags can also be added to recombinant proteins to provide convenient methods of isolation, for monitoring expression, and for monitoring cellular and subcellular localization, e.g., c-myc, hemagglutinin, or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo−5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells. Exemplary expression vectors include the pcDNA series, e.g., pcDNA3 (Invitrogen, Carlsbad, Calif.).

Some expression systems have markers for selection of stably transfected cell lines such as thymidine kinase, hygromycin B phosphotransferase, and dihydrofolate reductase. High-yield expression systems are also suitable, such as baculovirus vectors in insect cells, with a nucleic acid sequence encoding a fusion protein under the transcriptional control of the polyhedrin promoter or any other strong baculovirus promoter.

Elements that are typically included in expression vectors also include a replicon that functions in one or more host cells, a selective marker, e.g., a gene encoding antibiotic resistance, to permit selection of cells that harbor recombinant plasmids, and unique restriction sites in nonessential regions of the vector to allow insertion of recombinant sequences.

Standard transfection methods can be used to produce bacterial, mammalian, yeast, insect, or other cell lines that express large quantities of fusion proteins, which can be purified, if desired, using standard techniques. See, e.g., Colley et al. (1989) J. Biol. Chem. 264:17619-17622; and Guide to Protein Purification, in Methods in Enzymology, vol. 182 (Deutscher, ed.) 1990. Transformation of eukaryotic and prokaryotic cells is performed according to standard techniques. See, e.g., Morrison (1977) J. Bacteriol. 132:349-351; Clark-Curtiss et al. (1983) in Methods in Enzymology 101:347-362 (Wu et al., eds).

Any procedure for introducing exogenous nucleotide sequences into host cells can be used. These include, but are not limited to, the use of calcium phosphate transfection, DEAE-dextran-mediated transfection, polybrene, protoplast fusion, electroporation, lipid-mediated delivery (e.g., liposomes), microinjection, particle bombardment, introduction of naked DNA, plasmid vectors, viral vectors (both episomal and integrative) and any of the other well known methods for introducing cloned genomic DNA, cDNA, synthetic DNA or other exogenous genetic material into a host cell (see, e.g., Sambrook et al., supra). It is only necessary that the particular genetic engineering procedure used be capable of successfully introducing at least one gene into the host cell capable of expressing the protein of choice.

Conventional viral and non-viral based gene transfer methods can be used to introduce nucleic acids into mammalian cells or target tissues. Such methods can be used to administer nucleic acids to cells in vitro for screening as described herein. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For reviews of gene therapy procedures, see, for example, Anderson (1992) Science 256:808-813; Nabel et al. (1993) Trends Biotechnol. 11:211-217; Mitani etal. (1993) Trends Biotechnol. 11:162-166; Dillon (1993) Trends Biotechnol. 11: 167-175; Miller (1992) Nature 357:455-460; Van Brunt (1988) Biotechnology 6(10): 1149-1154; Vigne (1995) Restorative Neurology and Neuroscience 8:35-36; Kremer et al. (1995) British Medical Bulletin 51(1):31-44; Haddada et al., in Current Topics in Microbiology and Immunology, Doerfler and Böhm (eds), 1995; and Yu et al. (1994) Gene Therapy 1:13-26.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386; 4,946,787; and 4,897,355 and lipofection reagents are sold commercially (e.g., Transfectam™ and Lipofectin™). Cationic and neutral lipids that are suitable for efficient receptor-recognition lipofection of polynucleotides include those of Felgner, WO 91/17424 and WO 91/16024.

The preparation of lipid:nucleic acid complexes, including targeted liposomes such as immunolipid complexes, is well known to those of skill in the art. See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; and U.S. Pat. Nos. 4,186,183; 4,217,344; 4,235,871; 4,261,975; 4,485,054; 4,501,728; 4,774,085; 4,837,028 and 4,946,787.

The use of RNA or DNA virus-based systems for the delivery of nucleic acids take advantage of highly evolved processes for targeting a virus to specific cells in the body and trafficking the viral payload to the nucleus. Conventional viral-based systems for the delivery of ZFPs include retroviral, lentiviral, poxviral, adenoviral, adeno-associated viral, vesicular stomatitis viral and herpesviral vectors. Integration in the host genome is possible with certain viral vectors, including the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, allowing alteration and/or expansion of the potential target cell population. Lentiviral vectors are retroviral vector that are able to transduce or infect non-dividing cells and typically produce high viral titers. Selection of a retroviral gene transfer system would therefore depend on the target tissue. Retroviral vectors have a packaging capacity of up to 6-10 kb of foreign sequence and are comprised of cis-acting long terminal repeats (LTRs). The minimum cis-acting LTRs are sufficient for replication and packaging of the vectors, which can then be used to integrate an exogenous gene into a target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof. Buchscher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; and PCT/US94/05700).

Adeno-associated virus (AAV) vectors are also used to transduce cells with target nucleic acids, e.g., in the in vitro production of nucleic acids and peptides, and for in vivo and ex vivo gene therapy procedures. See, e.g., West et al. (1987) Virology 160:38-47; U.S. Pat. No. 4,797,368; WO 93/24641; Kotin (1994) Hum. Gene Ther. 5:793-801; and Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin, et al. (1984) Mol. Cell. Biol. 4:2072-2081; Hermonat et al. (1984) Proc. Natl. Acad. Sci. USA 81:6466-6470; and Samulski etal. (1989) J. Virol. 63:3822-3828.

Recombinant adeno-associated virus vectors based on the defective and nonpathogenic parvovirus adeno-associated virus type 2 (AAV-2) are a promising gene delivery system. Exemplary AAV vectors are derived from a plasmid containing the AAV 145 bp inverted terminal repeats flanking a transgene expression cassette. Efficient gene transfer and stable transgene delivery due to integration into the genomes of the transduced cell are key features for this vector system. Wagner et al. (1998) Lancet 351(9117):1702-3; and Kearns et al. (1996) Gene Ther. 9:748-55.

pLASN and MFG-S are examples are retroviral vectors that have been used in clinical trials. Dunbar et al. (1995) Blood 85:3048-305; Kohn et al. (1995) Nature Med. 1:1017-102; Malech et al. (1997) Proc. Natl. Acad. Sci. USA 94:12133-12138. PA317/pLASN was the first therapeutic vector used in a gene therapy trial. (Blaese et al. (1995) Science 270:475-480. Transduction efficiencies of 50% or greater have been observed for MFG-S packaged vectors. Ellem et al. (1997) Immunol Immunother. 44(1):10-20; Dranoff et al. (1997) Hum. Gene Ther. 1:111-2.

In applications for which transient expression is preferred, adenoviral-based systems are useful. Adenoviral based vectors are capable of very high transduction efficiency in many cell types and are capable of infecting, and hence delivering nucleic acid to, both dividing and non-dividing cells. With such vectors, high titers and levels of expression have been obtained. Adenovirus vectors can be produced in large quantities in a relatively simple system.

Replication-deficient recombinant adenovirus (Ad) vectors can be produced at high titer and they readily infect a number of different cell types. Most adenovirus vectors are engineered such that a transgene replaces the Ad E1 a, E1b, and/or E3 genes; these replication-defective vectors are propagated in human 293 cells that supply the required E1 functions in trans. Ad vectors can transduce multiple types of tissues in vivo, including non-dividing, differentiated cells such as those found in the liver, kidney and muscle. Conventional Ad vectors have a large carrying capacity for inserted DNA.

Packaging cells are used to form virus particles that are capable of infecting a host cell. Such cells include 293 cells, which package adenovirus, and Ψ2 cells or PA317 cells, which package retroviruses. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. Missing viral functions are supplied in trans, if necessary, by the packaging cell line. For example, AAV vectors used in gene therapy typically only possess ITR sequences from the AAV genome, which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment, which preferentially inactivates adenoviruses.

In many gene therapy applications, it is desirable that the gene therapy vector be delivered with a high degree of specificity to a particular tissue type. A viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751 reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., F_{ab} or Fᵥ) having specific binding affinity for virtually any chosen cellular receptor. Although the above description applies primarily to viral vectors, the same principles can be applied to non-viral vectors. Such vectors can be engineered to contain specific uptake sequences thought to favor uptake by specific target cells.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art. See, e.g., Freshney et al., Culture of Animal Cells, A Manual of Basic Technique, 3rd ed., 1994, and references cited therein, for a discussion of isolation and culture of cells from patients.

The use of ex vivo transfection methods to screen for transcriptional regulatory domains as disclosed herein will allow the development of custom-designed therapeutics. For example, if cells from a tumor biopsy are determined to exhibit overexpression of a particular gene, libraries (as disclosed herein) can be designed in which the DNA-binding portion is targeted to the overexpressed gene. The biopsy cells can then be cultured ex vivo and used to screen the libraries for peptide sequences that repress expression of the overexpressed gene. Similarly, pathological cells characterized by underexpression of a particular gene can be used to screen for transcriptional activators of the underexpressed gene.

In one embodiment, hematopoietic stem cells are used in ex vivo procedures for cell transfection and ex vivo gene therapy. The advantage to using stem cells is that they can be differentiated into other cell types in vitro, or can be introduced into a mammal (such as the donor of the cells) where they will engraft in the bone marrow. Methods for differentiating CD34+ stem cells in vitro into clinically important immune cell types using cytokines such a GM-CSF, IFN-γ and TNF-α are known. Inaba et al. (1992) J. Exp. Med. 176:1693-1702.

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells). See Inaba et al., supra.

Vectors (e.g., retroviruses, adenoviruses, liposomes, etc.) can be also administered directly to an organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there are a wide variety of suitable formulations of pharmaceutical compositions described herein. See, e.g., Remington's Pharmaceutical Sciences, 17th ed., 1989.

B. Delivery of Polypeptides Encoding Fusion Proteins

In certain embodiments, fusion proteins are administered directly to target cells. An important factor in the administration of polypeptides is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins, lipids and other compounds, which have the ability to translocate polypeptides across a cell membrane, have been described.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58. Prochiantz (1996) Curr. Opin. Neurobiol. 6:629-634. Another subsequence, the h (hydrophobic) domain of signal peptides, was found to have similar cell membrane translocation characteristics. Lin et al. (1995) J. Biol. Chem. 270:14255-14258.

Examples of peptide sequences which can be linked to a fusion polypeptide for facilitating its uptake into cells include, but are not limited to: an 11 amino acid peptide of the tat protein of HIV; a 20 residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et al. (1994) J. Biol. Chem. 269:10444); the h region of a signal peptide, such as the Kaposi fibroblast growth factor (K-FGF) h region (Lin et al., supra); and the VP22 translocation domain from HSV (Elliot et al. (1997) Cell 88:223-233). Other suitable chemical moieties that provide enhanced cellular uptake can also be linked, either covalently or non-covalently, to fusion polypeptides.

Toxin molecules also have the ability to transport polypeptides across cell membranes. Often, such molecules (called “binary toxins”) are composed of at least two parts: a translocation or binding domain and a separate toxin domain. Typically, the translocation domain, which can optionally be a polypeptide, binds to a cellular receptor, facilitating transport of the toxin into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci. USA. 89:10277-10281; and Novak et al. (1992) J. Biol. Chem. 267:17186-17193.

Such subsequences can be used to translocate polypeptides, including fusion polypeptides as disclosed herein, across a cell membrane. This is accomplished, for example, by derivatizing the fusion polypeptide with one of these translocation sequences, or by forming an additional fusion of the translocation sequence with the fusion polypeptide. Optionally, a linker can be used to link the fusion polypeptide and the translocation sequence. Any suitable linker can be used, e.g., a peptide linker.

A suitable polypeptide can also be introduced into an animal cell, preferably a mammalian cell, via liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell.

The liposome fuses with the plasma membrane, thereby releasing the compound into the cytosol. Alternatively, the liposome is phagocytosed or taken up by the cell in a transport vesicle. Once in the endosome or phagosome, the liposome is either degraded or it fuses with the membrane of the transport vesicle and releases its contents.

In current liposome delivery methods, the liposome ultimately becomes permeable and releases the encapsulated compound at the target tissue or cell. For systemic or tissue specific delivery, this can be accomplished, for example, in a passive manner wherein the liposome bilayer is degraded over time through the action of various agents in the body. Alternatively, active drug release involves using an agent to induce a permeability change in the liposome vesicle. Liposome membranes can be constructed so that they become destabilized when the environment becomes acidic near the liposome membrane. See, e.g., Proc. Natl. Acad. Sci. USA 84:7851 (1987); Biochemistry 28:908 (1989). When liposomes are endocytosed by a target cell, for example, they become destabilized and release their contents. This destabilization is termed fusogenesis. Dioleoylphosphatidylethanolamine (DOPE) is the basis of many “fusogenic” systems.

For use with the methods and compositions disclosed herein, liposomes typically comprise a fusion polypeptide as disclosed herein, a lipid component, e.g., a neutral and/or cationic lipid, and optionally include a receptor-recognition molecule such as an antibody that binds to a predetermined cell surface receptor or ligand (e.g., an antigen). A variety of methods are available for preparing liposomes as described in, e.g.; U.S. Pat. Nos. 4,186,183; 4,217,344; 4,235,871; 4,261,975; 4,485,054; 4,501,728; 4,774,085; 4,837,028; 4,235,871; 4,261,975; 4,485,054; 4,501,728; 4,774,085; 4,837,028; 4,946,787; PCT Publication No. WO 91/17424; Szoka et al. (1980) Ann. Rev. Biophys. Bioeng. 9:467; Deamer et al. (1976) Biochim. Biophys. Acta 443:629-634; Fraley, et al. (1979) Proc. NatL. Acad. Sci. USA 76:3348-3352; Hope et al. (1985) Biochim. Biophys. Acta 812:55-65; Mayer et al. (1986) Biochim. Biophys. Acta 858:161-168; Williams et al. (1988) Proc. Natl. Acad. Sci. USA 85:242-246; Liposomes, Ostro (ed.), 1983, Chapter 1); Hope et al. (1986) Chem. Phys. Lip. 40:89; Gregoriadis, Liposome Technology (1984) and Lasic, Liposomes: from Physics to Applications (1993). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it may be desirable to target a liposome using targeting moieties that are specific to a particular cell type, tissue, and the like. Targeting of liposomes using a variety of targeting moieties (e.g., ligands, receptors, and monoclonal antibodies) has been previously described. See, e.g., U.S. Pat. Nos. 4,957,773 and 4,603,044.

Examples of targeting moieties include monoclonal antibodies specific to antigens associated with a particular cell. Standard methods for coupling targeting agents to liposomes are used. These methods generally involve the incorporation into liposomes of lipid components, e.g., phosphatidylethanolamine, which can be activated for attachment of targeting agents, or incorporation of derivatized lipophilic compounds, such as lipid derivatized bleomycin. Antibody targeted liposomes can be constructed using, for instance, liposomes which incorporate protein A. See Renneisen et al. (1990) J. Biol Chem. 265:16337-16342 and Leonetti et al. (1990) Proc. Natl. Acad. Sci. USA 87:2448-2451.

Applications

Once a functional domain is identified, a recombinant molecule can be constructed in which sequences encoding the functional domain are placed in operative linkage with sequences encoding a targeted DNA-binding domain which binds a target sequence of interest, to generate a fusion protein capable of regulating a gene of choice. Thus, for example, to obtain a protein which regulates a therapeutically relevant gene in a particular cell type, one can assay a library, as disclosed herein, in that cell type, wherein the DNA-binding portion of the proteins encoded by the library is targeted to an easily-assayable reporter gene. Once a suitable regulatory domain is identified, it can be fused to a DNA-binding domain targeted to the therapeutically relevant gene (e.g., an engineered zinc finger protein) or, preferably, a recombinant molecule is constructed which contains sequences encoding the suitable regulatory domain in operative linkage with sequences encoding a DNA-binding domain targeted to the therapeutically relevant gene. Such a construct is then introduced into cells such that the fusion protein is expressed.

Functional domains, identified as disclosed herein, can be used to facilitate a number of processes involving transcriptional regulation. These processes include, but are not limited to, transcription, replication, recombination, repair, integration, maintenance of telomeres, processes involved in chromosome stability and disjunction, and maintenance and propagation of chromatin structures. Accordingly, regulatory domains obtained using the methods and compositions disclosed herein can be used to affect any of these processes, as well as any other process which can be influenced by a transcriptional regulatory domain's effect on gene expression and DNA binding proteins. As a result, expression of any gene in any cell in any organism can be modulated using transcriptional regulatory domains obtained according to the methods and compositions disclosed herein, including therapeutically relevant genes, genes of infecting microorganisms, viral genes, and genes whose expression is modulated in the process of target validation. Such genes include, but are not limited to, Wilms' third tumor gene (WT3), vascular endothelial growth factor (VEGF), VEGF receptors flt and flk, CCR-5, low density lipoprotein receptor (LDLR), estrogen receptor, HER-2/neu, BRCA-1, BRCA-2, phosphoenolpyruvate carboxykinase (PEPCK), CYP7, fibrinogen, apolipoprotein A (ApoA), apolipoprotein B (ApoB), renin, phosphoenolpyruvate carboxykinase (PEPCK), CYP7, fibrinogen, nuclear factor κB (NF-κB), inhibitor of NF-κB (I-κB), tumor necrosis factors (e.g., TNF-α, TNF-β), interleukin-1 (IL-1), FAS (CD95), FAS ligand (CD95L), atrial natriuretic factor, platelet-derived factor (PDF), amyloid precursor protein (APP), tyrosinase, tyrosine hydroxylase, β-aspartyl hydroxylase, alkaline phosphatase, calpains (e.g., CAPN10) neuronal pentraxin receptor, adriamycin response protein, apolipoprotein E (apoE), leptin, leptin receptor, UCP-1, IL-1, IL-1 receptor, IL-2, IL-3, IL-4, IL-5, IL-6, IL-12, IL-15, interleukin receptors, G-CSF, GM-CSF, colony stimulating factor, erythropoietin (EPO), platelet-derived growth factor (PDGF), PDGF receptor, fibroblast growth factor (FGF), FGF receptor, PAF, p16, p19, p53, Rb, p²¹, myc, myb, globin, dystrophin, eutrophin, cystic fibrosis transmembrane conductance regulator (CFTR), GNDF, nerve growth factor (NGF), NGF receptor, epidermal growth factor (EGF), EGF receptor, transforming growth factors (e.g., TGF-α, TGF-β), fibroblast growth factor (FGF), interferons (e.g., IFN-α, IFN-β and TFN-γ), insulin-related growth factor-1 (IGF-1), angiostatin, ICAM-1, signal transducer and activator of transcription (STAT), androgen receptors, e-cadherin, cathepsins (e.g., cathepsin W), topoisomerase, telomerase, bcl, bcl-2, Bax, T Cell-specific tyrosine kinase (Lck), p38 mitogen-activated protein kinase, protein tyrosine phosphatase (hPTP), adenylate cyclase, guanylate cyclase, α7 neuronal nicotinic acetylcholine receptor, 5-hydroxytryptamine (serotonin)-2A receptor, transcription elongation factor-3 (TEF-3), phosphatidylcholine transferase, fitz, PTI-1, polygalacturonase, EPSP synthase, FAD2-1, Δ-9 desaturase, Δ-12 desaturase, Δ-15 desaturase, acetyl-Coenzyme A carboxylase, acyl-ACP thioesterase, ADP-glucose pyrophosphorylase, starch synthase, cellulose synthase, sucrose synthase, fatty acid hydroperoxide lyase, and peroxisome proliferator-activated receptors, such as PPAR-γ2.

Expression of human, mammalian, bacterial, fungal, protozoal, Archaeal, plant and viral genes can be modulated; viral genes include, but are not limited to, hepatitis virus genes such as, for example, HBV-C, HBV-S, HBV-X and HBV-P; and HIV genes such as, for example, tat and rev. Modulation of expression of genes encoding antigens of a pathogenic organism can be achieved using the disclosed methods and compositions. Modulation of expression of a purinergic cell-surface receptor gene such as, for example, P2X7 can be used for identification of transcriptional repression domains.

Additional genes include those encoding cytokines, lymphokines, interleukins, growth factors, mitogenic factors, apoptotic factors, cytochromes, chemotactic factors, chemokine receptors (e.g., CCR-2, CCR-3, CCR-5, CXCR-4), phospholipases (e.g., phospholipase C), nuclear receptors, retinoid receptors, organellar receptors, hormones, hormone receptors, oncogenes, tumor suppressors, cyclins, cell cycle checkpoint proteins (e.g., Chk1, Chk2), senescence-associated genes, immunoglobulins, genes encoding heavy metal chelators, protein tyrosine kinases, protein tyrosine phosphatases, tumor necrosis factor receptor-associated factors (e.g., Traf-3, Traf-6), apolipoproteins, thrombic factors, vasoactive factors, neuroreceptors, cell surface receptors, G-proteins, G-protein-coupled receptors (e.g., substance K receptor, angiotensin receptor, α- and β-adrenergic receptors, serotonin receptors, and PAF receptor), muscarinic receptors, acetylcholine receptors, GABA receptors, glutamate receptors, dopamine receptors, adhesion proteins (e.g., CAMs, selecting, integrins and immunoglobulin superfamily members), ion channels, receptor-associated factors, hematopoietic factors, transcription factors, and molecules involved in signal transduction. Expression of disease-related genes, and/or of one or more genes specific to a particular tissue or cell type such as, for example, brain, muscle, heart, nervous system, circulatory system, reproductive system, genitourinary system, digestive system and respiratory system can also be modulated.

Thus, regulatory domains obtained according to the methods and compositions disclosed herein can be used in processes such as, for example, therapeutic regulation of disease-related genes, engineering of cells for manufacture of protein pharmaceuticals, pharmaceutical discovery (including target discovery, target validation and engineering of cells for high throughput screening methods) and plant agriculture.

### EXAMPLES

The following examples are presented as illustrative of, but not limiting, the claimed subject matter.

### Example 1

Library Generation

Degenerate oligonucleotides encoding a peptide library are synthesized. One or more primers are annealed to the degenerate oligonucleotide, and double-stranded DNA is synthesized using a DNA polymerase such as, for example, E. coli DNA polymerase, E. coli DNA polymerase Klenow fragment, T4 DNA polymerase, T7 DNA polymerase or Sequenase 2.0 (USB, Cleveland, Ohio.). Alternatively, a cDNA library is constructed, using methods that are well-known to those of skill in the art. See, for example, Sambrook et al., supra and Ausubel et al., supra. The double-stranded product (oligonucleotide or cDNA) is introduced into a vector to generate a population of constructs in which a targeted DNA-binding domain (e.g., a zinc finger binding domain) is fused to a plurality of randomized peptide sequences or cDNA sequences. The library sequences (random or cDNA) can be introduced into a vector already comprising a sequence encoding a targeted ZFP, or they can be cloned into a vector, and a sequence encoding a targeted ZFP can subsequently be introduced into the vector. Generation of these constructs is accomplished by known recombinant methods such as, for example, digestion with restriction enzymes, end modification, and ligation.

Ligation mixtures are used to transform prokaryotic host cells (e.g., E. coli). The efficiency of transformation is determined and the total number of clones is calculated to determine the complexity of the library. Plasmid DNA is then isolated from the all of the members of the library and is introduced into host cells (preferably mammalian) comprising a reporter. The reporter can, for example, be encoded by an endogenous gene or by an exogenous reporter construct. In the latter case, the exogenous reporter construct can be transiently transfected into the host cells, or stably maintained (e.g. on an episome or by integration).

A volume of the library stock is grown and prepared for transient or stable transfection into a selected host cell comprising a reporter. Transfected cells are analyzed, for example, by FACS, to estimate transfection efficiency and to establish the sorting window. Transfected cells are then sorted, for example, by FACS or magnetic cell sorting and positive cells (i.e., cells in which expression of the reporter gene is modulated) are collected. Clones are obtained from positive cells, are amplified and their nucleotide sequences are determined. Sequences of various individual clones from the amplified cells indicate no sequence bias. Plasmids are recovered from the clones by standard techniques; alternatively, sequences encoding putative functional domains can be recovered by PCR, using primers that bind to flanking sequences in the vector construct.

### Example 2

Generation of Stable Reporter-Containing Cell Lines

Constructs containing a selected reporter are constructed and introduced into an appropriate cell line. The constructs optionally contain sequences encoding a selectable marker such as, for example, neomycin resistance. In this case, cell lines containing stably integrated reporter constructs are selected using neomycin (G418). Cells are transfected by lipofection with plasmids encoding each reporter and are grown in Dulbecco's modified Eagle medium (DMEM) containing 10% fetal bovine serum and 1 mg/ml neomycin. The medium is changed regularly. Following serial dilution in 96-well plates and 3-4 weeks of growth, single colonies are chosen and tested for expression of the reporter.

### Example 3

Functional Assay for High-Throughput Screening

This example describes the use of the P2X7 gene as a reporter gene for high-throughput screening of peptide or cDNA libraries to identify repressor peptides/proteins.

The P2X7 purinoceptor is a ligand-gated ion channel expressed on the surface of cells of immune and hematopoietic origin. When activated by low concentrations of ATP or the more potent benzoylbenzoyl ATP (BzATP), P2X7 acts as a non-specific channel for small cations. Higher concentrations of agonists stimulate P2X7 to open larger pores permeable to fluorescent DNA-binding dyes such as Ethidium Bromide (EtBr) and Yo-Pro-1 (a less cell-toxic dye).

Prolonged activation of P2X7 results in cell death due specifically to activity of this receptor. These properties of the P2X7 receptor are described in Coutinho-Silva et al. 1999, supra; Virgino et al. 1999, supra; Le Feuvre et al. 2002, supra; and Brough et al. 2002, supra.

Taking advantage of these properties, the P2X7 gene can be used as a reporter for high-throughput functional screening for repressor domains. Indeed, measurement of DNA-binding dye uptake in cells treated with ATP or BzATP using flow cytometry is a standard method for monitoring expression of the P2X7 receptor. Nihei et al. (2000) Mem Inst Oswaldo Cruz May-June; 95(3):415-428.

To test the suitability of the P2X7 gene as a reporter for high-throughput screening for repression domains, mouse Neuro-2A cells (expressing P2X7) were transiently transfected with a plasmid expressing a zinc finger DNA-binding domain targeted to the P2X7 gene (ZFP #5493) linked to a KOX1 repression domain. Control Neuro-2A cells were transfected with a vector lacking both library sequences and ZFP-encoding sequences (i.e., empty vector). Cells were treated for 10 minutes with different concentrations of BzATP (ranging from 0 to 1 mM), and EtBr was then added to a final concentration of 10 μM. After 20 minutes, cells were harvested by centrifugation, washed in PBS and analyzed by FACS.

The results of the FACS analysis indicated that low concentrations of BzATP (up to 0.04 mM) did not induce significant uptake of EtBr in Neuro-2A cells. However, higher concentrations induced significant accumulation of EtBr inside cells. In addition, cells transfected with a construct encoding a ZFP linked to a repressor domain accumulated 2-3-fold less EtBr, compared to control cells transfected with empty vector. These results indicate that cells expressing lower levels of P2X7 (due to the action of a repression domain directed to the P2X7 target gene by the ZFP) can be separated by sorting on the basis of lower uptake of EtBr (or any other DNA-binding dyes).

### Example 4

Selection Method for Repression Domains

The ability of enhanced P2X7 activity to kill cells (following prolonged agonist activation) represents another approach for functional high-throughput screening. In this approach, a library of constructs encoding putative repression domains, targeted to the P2X7 gene, is introduced into cells in which P2X7 activity is agonized, and surviving cells are selected.

To test this selection method, Neuro-2A cells were transiently transfected with empty vector (see Example 3 supra) or with a plasmid expressing a KOX1 repressor domain linked to the P2X7-targeted zinc finger binding domain ZFP #5493. 48 hours after transfection, cells were detached from the plate with Trypsin-EDTA, collected by centrifugation and resuspended in HKS assay buffer (125 mM KC1, 1 mM EDTA, 5 mM glucose, 20 mM HEPES, pH 7.4 adjusted with KOH). Resuspended cells were mixed with BzATP (final concentrations ranging from 0 to 1 mM) and plated onto 6 well plates (1×10⁵ cells/well in 1 ml HKS). After a 2h incubation at 37° C. in a cell incubator, cells were washed with PBS, and growth medium (minimal essential medium with Earle's salts and non-essential amino acids, adjusted to contain 2 mM L-glutamine, 1.5 g/L Na bicarbonate, 1.0 mM Na pyruvate and 10% fetal bovine serum) was added to plates.

Two to three days after transfection, the following observations were made. Treatment with relatively low concentrations of BzATP (>20 μM) resulted in some death of cells transfected with either empty vector or with the ZFP-KOX construct. However, after treatment of cells with BzATP concentrations >100 μM, almost no survival of cells transfected with empty vector was observed, while 10-100-fold higher survival was observed for cells transfected with the ZFP-KOX construct. These results indicate that BzATP selection is a simple method for screening for repressor domains targeted by a ZFP to the P2X7 reporter gene.

Thus, the P2X7 gene can be used as a reporter to screen combinatorial peptide or cDNA libraries (linked to a targeted DNA-binding domain such as, for example, a ZFP) for peptides/protein domains having transcriptional repression activity. Libraries are cloned into plasmid or viral vectors and transfected or transduced, accordingly, into a recipient cell line comprising a P2X7 reporter, which may be simply the endogenous chromosomal P2X7 gene (in cells of immune origin, for example) or which can be an exogenous reporter gene (comprising a desired promoter linked to the P2X7 cDNA). Exogenous genes can be transiently transfected or stably integrated into chromosomal DNA of cells lacking expression of their own chromosomal P2X7 gene (for example human kidney 293 cells).

### Example 5

Delivery of Libraries into Mammalian Cells and Recovery of Transcriptional Effector Peptides/Proteins

There are two general methods for delivery of DNA into mammalian cells: transfection and transduction.

For transfection, double-stranded randomized oligonucleotides (encoding randomized peptides) or cDNA is cloned into a plasmid encoding a targeted ZFP (to create in-frame fusion proteins).

Plasmid ligation mixture is used to transform E. coli cells. Colonies are collected (washed using LB medium) from the plates, and total plasmid DNA (i.e., a library) is isolated. Several single clones are also isolated to confirm correct reading frame by sequencing. The libraries are transiently transfected into recipient cells comprising an appropriate reporter (the expression of which can be monitored, for example, by FACS).

Initially, transfected cells are analyzed by FACS to establish the sorting window. Then, transfected cells are sorted by FACS and positive cells are collected. In assaying for a repression domain, positive cells are those expressing the lowest amounts of reporter; while, in assaying for an activation domain, positive cells are those expressing the highest amounts of reporter. Total DNA is isolated from positive cells and used to transform E. coli cells to recover plasmid DNA. This DNA can be used for several addition rounds of selection (transfection—sorting—plasmid recovery), or single clones can be analyzed by nucleotide sequencing.

In the case of transduction, DNA libraries (randomized peptides or cDNA fused in-frame to a targeted DNA-binding domain) are ligated into, e.g., a retroviral vector. The ligation mixture is used to transform E. coli cells. Total plasmid DNA from the transformed E. coli cells is isolated and used to transfect a packaging cell line. After 2-3 days of incubation, supernatant is collected from the transfected packaging cells and retroviral particles are purified, e.g., by filtration. The retroviral particles are used to infect cells comprising the reporter gene, and 3-4 days after infection, when retroviral DNA (converted from viral RNA) has been integrated into chromosomal DNA, cells are sorted by FACS, as described above. In contrast to transient transfection, recovery of library DNA is performed by PCR or RT-PCR, and the nucleotide sequences of PCR products are determined.

### Example 6

Repression of P2X7 Expression

This example demonstrates that, in cells transduced with a retrovirus vector whose genome encodes a ZFP/repression domain fusion targeted to the P2X7 gene, P2X7 mRNA levels were reduced approximately 15-fold, compared to non-transduced cells. In addition, functional assays for cell survival and dye uptake indicated greatly reduced P2X7 function.

Sequences encoding the P2X7-targeted, six-finger ZFP #5493 (see Example 3) fused to a KOX-1 repression domain, were subcloned into the retroviral vector pLNCX-2, in which their expression was controlled by the CMV promoter. The target DNA sequence, to which ZFP#5493 binds is GATGGGTCTGAGTGGGGG (SEQ ID NO: 1) The amino acid sequences of the recognition regions (positions −1 through +6, with respect to the start of the alpha helix of each zinc finger) of the zinc fingers in 5493, are as follows:

| | | |
|---|---|---|
| Finger 1: | RSDHLSN | (SEQ ID NO:2) |
| Finger 2: | RSDHRTN | (SEQ ID NO:3) |
| Finger 3: | RSDNLST | (SEQ ID NO:4) |
| Finger 4: | RSHDRTK | (SEQ ID NO:5) |
| Finger 5: | RSDHLST | (SEQ ID NO:6) |
| Finger 6: | TNSNRTK | (SEQ ID NO:7) |

Neuro-2A cells were infected with the virus and, 5 days after infection, cells were treated with 0.1 mM BzATP for two hours as described in Example 4. Approximately three days later, survival was measured. Of the cells transfected with the ZFP/KOX-1-expressing virus, 5-10% survived BzATP treatment; this represented an approximately 100-fold greater survival level than that of untransfected cells. Thus, expression of P2X7 was significantly reduced by the ZFP/KOX-1 fusion protein. After an additional 7 days, ZFP/KOX-1-expressing cells were treated again with 0.1 mM or 0.5 mM BzATP. Almost all cells survived, suggesting that the cells were not expressing the P2X7 receptor (or were expressing very low amounts).

ZFP/KOX-1-expressing cells were also assayed for P2X7 activity by measuring their ability to take up the DNA-binding dye YO-PRO-1, as follows. Seven days after the initial BzATP treatment, pools of clones containing 2-5×10⁵ cells were incubated with 0.2 mM BzATP for 10 minutes in HKS Buffer (125 mM KCl, 1 mM EDTA, 5 mM glucose, 20 mM HEPES-KOH, pH 7.4). YO-PRO-1 (Molecular Probes, Inc., Eugene, Oreg.) was added to a final concentration of 5 μM. After an additional 25 min incubation in the presence of the dye, the cells were washed with PBS, collected by centrifugation, resuspended in PBS, and analyzed by FACS on a Beckman-Coulter EPICS XL flow cytometer.

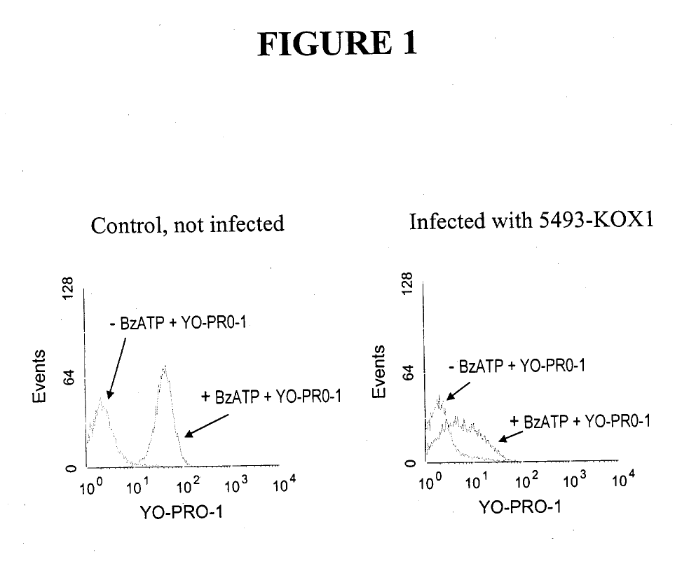

The results of the flow cytometric analysis, shown in FIG. 1, indicate that, in the presence of BzATP (i.e., with an activated P2X7 gene), cells take up substantial amounts of YO-PRO-1 (left panel); while cells expressing a ZFP/repression domain fusion, targeted to the P2X7 gene, take up much lower amounts of YO-PRO-1 in the presence of BzATP (right panel). Thus, the targeted ZFP/KOX-1 fusion protein has repressed the expression of the P2X7 gene. Accordingly, decrease in the ability to take up YO-PRO-1 and related dyes can be used as an assay for potential repression domains, by fusing the potential repression domain to a P2X7-targeted ZFP and measuring dye uptake in the presence of BzATP.

Four days after the survival and dye-uptake assays were conducted, expression of P2X7 mRNA levels were analyzed by real-time PCR (TaqMan®). GAPDH mRNA levels were used as a normalization standard. Primer and probe sequences for P2X7 and GAPDH are shown in Table 1. RNA was extracted using the High pure RNA Isolation kit (Roche Diagnostics, Indianapolis, Ind.).

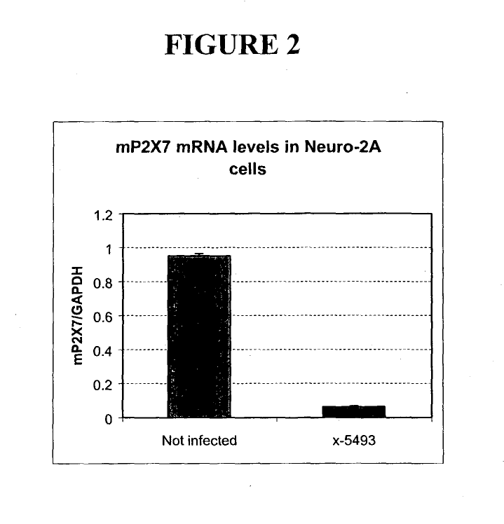

The results of the RNA analysis, shown in FIG. 2, indicate a 15-fold repression of P2X7 mRNA levels in cells that had been infected with the ZFP/KOX-1-expressing virus. Thus, a P2X7-targeted ZFP, fused to a repression domain, was able to repress transcription of the P2X7 gene.

TABLE 1

Primers and Probes

Sequence

| | | |
|---|---|---|
| P2X7 forward primer | 5′-CTGTACCAGCGGAAAGAGCCT-3′ | (SEQ ID NO:8) |
| P2X7 reverse primer | 5′-CCCTGCAAAGGGAAGGTGTAG-3′ | (SEQ ID NO:9) |
| P2X7 probe | 5′-TGCACACCAAGGTCAAAGGCATAGCA-3′ | (SEQ ID NO:10) |
| GAPDH forward primer | 5′-CCCATGTTTGTGATGGGTGTG-3′ | (SEQ ID NO:11) |
| GAPDH reverse primer | 5′-TGGCATGGACTGTGGTCATGA-3′ | (SEQ ID NO:12) |
| GAPDH probe | 5′-ATCCTGCACCACCAACTGCTTAGC-3′ | (SEQ ID NO:13) |

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of identifying a transcriptional regulatory peptide, the method comprising the following steps:
(a) contacting a population of cells comprising a reporter gene with a library of expression vectors, wherein each expression vector encodes a protein comprising:
(i) a first domain comprising a DNA-binding domain which binds to the reporter gene, and
(ii) a second domain;
(b) identifying a cell in which expression of the reporter gene is modulated; and
(c) characterizing the vector from said cell so as to identify the second domain, wherein said second domain encodes a transcriptional regulatory peptide.

2. The method of claim 1, wherein the DNA-binding domain comprises at least one zinc finger.

3. The method of claim 2, wherein the at least one zinc finger is an engineered zinc finger.

4. The method of claim 1, wherein the reporter gene is an endogenous gene.

5. The method of claim 4, wherein the reporter gene encodes a molecule that is expressed on the cell surface.

6. The method of claim 1, wherein the reporter gene is an exogenous gene.

7. The method of claim 6, wherein the reporter gene encodes a molecule that is expressed on the cell surface.

8. The method of claim 6, wherein the reporter gene is stably maintained in the cells.

9. The method of claim 8, wherein the reporter gene is integrated into the cellular genome.

10. The method of claim 6, wherein the reporter gene is transiently transfected into the cells.

11. The method of claim 1 wherein identifying comprises subjecting the cells to fluorescence-activated cell sorting (FACS) analysis.

12. The method of claim 1, wherein the reporter gene is the P2X7 gene.

13. The method of claim 1, wherein expression of the reporter gene is modulated in a plurality of cells and the vector in each of the cells is characterized.


SEQUENCE LISTING (13 sequences)

SEQ ID NO: 1 - DNA, 18 bp, mammalian
gatgggtctgagtggggg

SEQ ID NO: 2 - protein, 7 aa, Artificial
  Zinc Finger 1 in 5493
RSDHLSN

SEQ ID NO: 3 - protein, 7 aa, Artificial
  Zinc Finger 2 in 5493
RSDHRTN

SEQ ID NO: 4 - protein, 7 aa, Artificial
  Zinc Finger 3 in 5493
RSDNLST

SEQ ID NO: 5 - protein, 7 aa, Artificial
  Zinc Finger 4 in 5493
RSHDRTK

SEQ ID NO: 6 - protein, 7 aa, Artificial
  Zinc Finger 5 in 5493
RSDHLST

SEQ ID NO: 7 - protein, 7 aa, Artificial
  Zinc Finger 6 in 5493
TNSNRTK

SEQ ID NO: 8 - DNA, 21 bp, mammalian
ctgtaccagcggaaagagcct

SEQ ID NO: 9 - DNA, 21 bp, mammalian
ccctgcaaagggaaggtgtag

SEQ ID NO: 10 - DNA, 26 bp, mammalian
tgcacaccaaggtcaaaggcatagca

SEQ ID NO: 11 - DNA, 21 bp, mammalian
cccatgtttgtgatgggtgtg

SEQ ID NO: 12 - DNA, 21 bp, mammalian
tggcatggactgtggtcatga

SEQ ID NO: 13 - DNA, 24 bp, mammalian
atcctgcaccaccaactgcttagc